(19) United States
(12) Patent Application Publication
(10) Pub. No.: US 2021/0259563 A1
(43) Pub. Date: Aug. 26, 2021

(54) SYSTEM AND METHOD FOR HETEROGENOUS DATA COLLECTION AND ANALYSIS IN A DETERMINISTIC SYSTEM

(71) Applicant: MINDMAZE HOLDING SA, Lausanne (CH)

(72) Inventors: Tej TADI, Lausanne (CH); Gabriel GANNE, Lausanne (CH); Shashi BOBBA, Lausanne (CH); Nicolas BOURDAUD, Lausanne (CH)

(21) Appl. No.: 17/261,980

(22) PCT Filed: Apr. 5, 2019
(86) PCT No.: PCT/IB2019/052833

§ 371 Date: Jan. 21, 2021

Related U.S. Application Data
(60) Provisional application No. 62/653,642, filed on Apr. 6, 2018.

(51) Int. Cl. A61B 5/0205; G16H 40/67; G16H 40/40; G01P 13/00; A61B 5/11; A61B 5/0533; A61B 5/318; A61B 5/369; A61B 5/389; A61B 5/242

(52) U.S. Cl. CPC A61B 5/0205; G16H 40/67; G16H 40/40; G01P 13/00; A61B 5/11; G06F 13/20; A61B 5/318; A61B 5/369; A61B 5/389; A61B 5/242; A61B 5/0533

(57) ABSTRACT

A system and method for heterogenous data collection and analysis, that supports collection of such data from a plurality of sensors.

### FIELD OF THE INVENTION

The present invention is of a system and method for heterogenous data collection and analysis, and in particular, of such a system and method that supports collection of such data from a plurality of sensors.

### BACKGROUND OF THE INVENTION

Collecting data from biological and other sensors in real time is difficult. The increasing complexity of sensors and the increased demands for medical data have led to problems in developing systems that can handle these real time demands. Cost also plays an important part; more complex systems can be constructed at increased cost, but such increased cost may not be feasible or supportable.

As the general population ages in many countries, medical systems seek to deliver improved care at reduced cost. This requires an increased application of complex technologies but does not leave much room for spending increased amounts on these technologies.

Currently there are no suitable solutions to this problem. Instead, medical systems rely on human personnel to overcome these problems, by substituting manual labor for full automation.

### BRIEF SUMMARY OF THE INVENTION

The present invention, in at least some embodiments, is of a system and method for heterogenous data collection and analysis, that supports collection of data, preferably including medical data, from a plurality of sensors.

According to at least some embodiments, the system features a plurality of sensors for obtaining various types of data, including without limitation biosignal data and/or motion data in relation to a subject. The sensors are preferably arranged in a deterministic architecture. The deterministic architecture preferably also features a plurality of data consumers, which receive the sensor data. The components of the system preferably communicate through a communication channel, according to a predetermined window function, in which different sensors transmit their data at a predefined window. The window may be determined according to time, such that each sensor periodically has a set time period in which it may transmit data. The window may be determined according to a frequency division, in which the sensor is able to transmit data only at a specific frequency. A combination may also be used.

More preferably a plurality of nodes is provided, each node supporting data transmission from a particular sensor. Each node preferably features a processing unit for processing the data before transmission on the communication channel.

Preferably there is a data abstraction layer that abstracts the data before it is provided to the data consumers. This data abstraction layer enables a variety of different types of sensors to provide data, without requiring a data consumer to be able to specifically communicate with each type of sensor. The data abstraction layer may be considered to fulfill at least some of the functions of a driver, to support communication between the nodes and the data consumers.

The communication channel may comprise a bus, for example on a chip. The communication channel may also operate across a plurality of separate hardware components, for example a plurality of chips, a plurality of devices, a plurality of separate processing units (whether data providers or consumers) and the like. The present invention is not intended to be limited to implementation on a single chip.

If implemented across a plurality of separate hardware components, preferably the communication channel operates across the separate hardware components according to a deterministic architecture. In such a deterministic architecture, preferably communication between hardware components is determined according to a plurality of predetermined, deterministic functions. Some non-limiting examples of such functions include determining the window for communication, constraints on the components, requirements of the components, programs which are to process and/or consume data, and so forth.

According to at least some embodiments of the present invention, there is provided an operating system for controlling the process of obtaining data in real time from a plurality of sensors, said plurality of sensors including at least biosignal sensors, for consumption by one or more data consumers. The operating system features a plurality of predetermined functions for controlling a predetermined architecture. The sensors communicate with the one or more data consumers through a predetermined communication channel. The predetermined architecture determines the interactions of the sensors, the one or more data consumers and the communication channel. At the time of initiation of activity, the operating system determines the boot process, through which the necessary programs are loaded, the communication channel is activated, and the sensors and data consumer(s) are able to communicate through the communication channel.

Sensors as described herein include but are not limited to biosignal sensors, motion tracking sensors and additional subject related sensors. Non-limiting examples of biosignal sensors include EEG, EKG, GVR (galvanic skin response), EMG, and audio sensors. Non-limiting examples of motion tracking sensors include inertial sensors, IMU, optical sensors, one or more gyroscopes, one or more accelerometers, one or more magnetometers, and depth sensors. Non-limiting examples of subject related sensors include optical sensors and complete sensor systems, including without limitation the MindMotion Pro device of MindMaze SA (Lausanne, Switzerland).

Implementation of the method and system of the present invention involves performing or completing certain selected tasks or steps manually, automatically, or a combination thereof. Moreover, according to actual instrumentation and equipment of preferred embodiments of the method and system of the present invention, several selected steps could be implemented by hardware or by software on any operating system of any firmware or a combination thereof. For example, as hardware, selected steps of the invention could be implemented as a chip or a circuit. As software, selected steps of the invention could be implemented as a plurality of software instructions being executed by a computer using any suitable operating system. In any case, selected steps of the method and system of the invention could be described as being performed by a data processor, such as a computing platform for executing a plurality of instructions.

Although the present invention is described with regard to a “computing device”, a “computer”, or “mobile device”, it should be noted that optionally any device featuring a data processor and the ability to execute one or more instructions may be described as a computer, including but not limited to any type of personal computer (PC), a server, a distributed server, a virtual server, a cloud computing platform, a cellular telephone, an IP telephone, a smartphone, or a PDA (personal digital assistant). Any two or more of such devices in communication with each other may optionally comprise a “network” or a “computer network”.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in order to provide what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. In the drawings:

FIGS. 1A and 1B show exemplary data flows from the patient's actions and biological activity in accordance with embodiments;

FIG. 2A shows a schematic of a non-limiting, exemplary system for receiving data from a plurality of sensors and distributing it to a plurality of data consumers in accordance with embodiments;

FIG. 2B shows a flow of a non-limiting, exemplary method for providing data to a plurality of networked nodes through a communication channel in accordance with embodiments;

FIGS. 3A-3C show schematics of a non-limiting, exemplary system for determining the architecture of a platform for supporting data collection and provisioning in accordance with embodiments;

FIGS. 4A and 4B relate to schematics of non-limiting, exemplary systems for providing an operating system according to at least some embodiments of the present invention in accordance with embodiments;

FIG. 5 relates to a flow of a non-limiting, exemplary method for obtaining biosignal data and providing the data to one or more consuming applications in accordance with embodiments;

FIG. 6 relates to a schematic of an exemplary implementation of a controller in a functional system in accordance with embodiments;

FIG. 7A relates to a schematic of an exemplary implementation of a controller as a chip in accordance with embodiments;

FIG. 7B shows a schematic of an optional additional implementation of the chip of FIG. 7, with the addition of edge preprocessing of the data signals, in accordance with embodiments;

FIG. 8 shows a schematic of an exemplary implementation of a chip as a 3D (three dimensional) chip in accordance with preferred embodiments; and

FIGS. 9A and 9B show schematics of an exemplary implementation of a system for obtaining sensor data for medical analysis in accordance with embodiments.

### DESCRIPTION OF AT LEAST SOME EMBODIMENTS

The present invention, in at least some embodiments, is of a system and method for cyber-medical data collection and analysis. The system abstracts the interactions of lower level hardware components, to enable a system of multiple hardware components to interact. Preferably the system architecture includes a deterministic component, for supporting efficient communication of the hardware components. Efficient communication preferably includes both resource-efficient and time-efficient communication. For example, such efficiencies may enable the system to be implemented as a chip, on a low computational resource device (such as a mobile telephone for example), through a network of such chips or low level computational resource devices and the like.

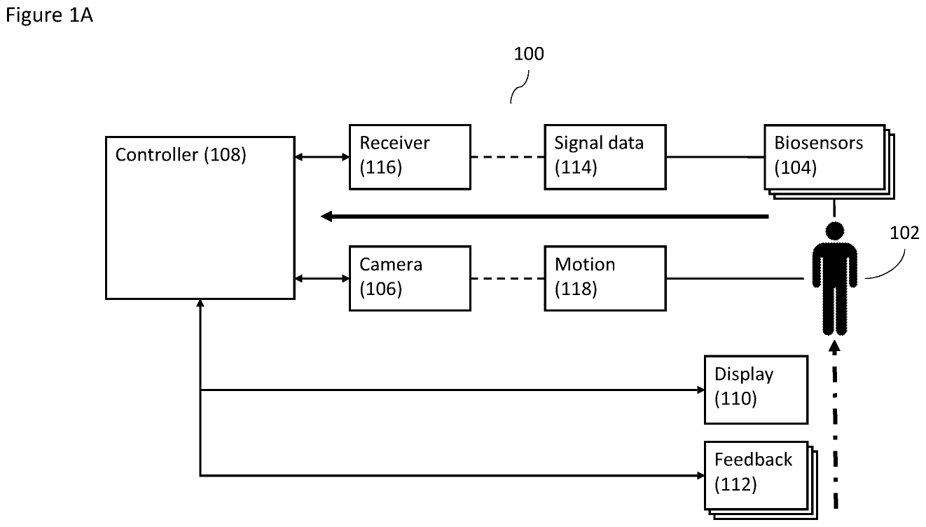

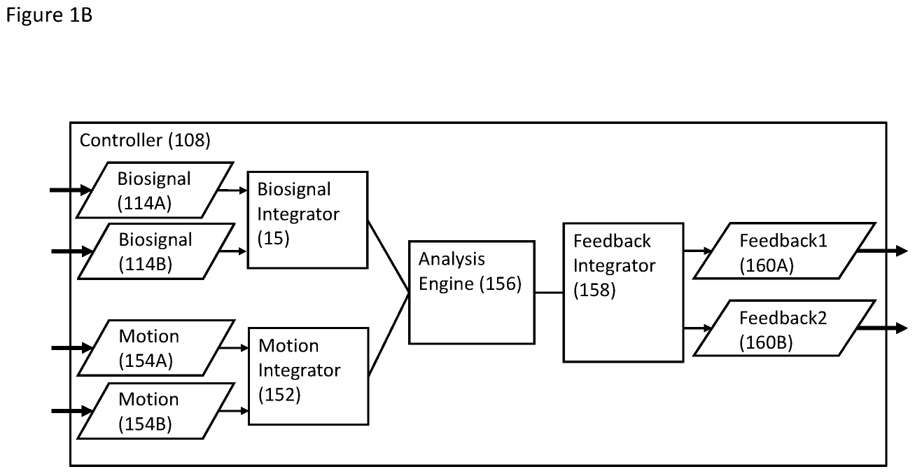

Turning now to the drawings, FIGS. 1A and 1B show data flows from the patient's actions and biological activity in accordance with embodiments. The patient 102 is shown at the center of the data flows and feedback. The actions and biological activity of patient 102 are measured by various devices, such as one or more biosensors 104 and/or one or more cameras 106. The measurements are provided to the controller 108 for analysis. The results of the analysis return to the patient 102 in the form of a changed display 110 and various types of feedback 112. Each type of data preferably has a corresponding device to measure it.

For example, a device to measure the actions of patient 102 could include one or more cameras 106, including without limitation an optical camera, a depth sensor or a combination thereof, to capture data related to the motions 118 of patient 102. A device to measure biological activity could include any type of suitable biosensor 104, including but not limited to a plurality of EEG or EMG sensors, for example. Signal data 114 from such sensors 104 is preferably received by an appropriate receiver 116. The terms “signal data” and “signals” are used interchangeably.

The patient 102 then preferably receives feedback from a patient-facing device. Such patient-facing devices preferably include but are not limited to a visual display 110 for the patient 102, and/or one or more various types of feedback 112, including without limitation haptic feedback, audio feedback and the like.

Each such device preferably has at least one corresponding connection to the controller 108. The connection is preferably bi-directional but may also be uni-directional. Also each patient-facing device preferably has a corresponding connection to the controller. Again, the connection is preferably bi-directional but may also be uni-directional. Each connection may be configured as a virtual representation on, or alternatively, a suitable interface to, the controller 108.

FIG. 1B shows an optional, exemplary configuration of the controller 108, featuring a plurality of connections, at least one such connection corresponding to each device that provides an incoming input and each device that provides an outgoing output. The inputs are preferably integrated, first at the level of the type of input (biosignal data, for example, etc.) and then at an overall analysis level.

This configuration makes it easy to add different types of input devices. Each level of integration also makes it possible to add context awareness. In this example, integration of various signals may lead to context awareness, but so can the addition of outside information about the patient (such as the patient's medical condition, physical condition and so forth). Context awareness may also relate to data patterns which indicate an increase or decrease in patient engagement and motivation.

If information from a particular input device is not needed at some point in time, or to avoid excess computational complexity, at each level of integration it is possible to ignore incoming information.

The higher level analysis also supports derivation of abilities and challenges which relate to patient systems, even if such systems cannot be directly measured by a single device. For example, the cognitive ability of a patient can be determined according to their ability to perform a particular task—which can require input from multiple devices—as well as their reaction to the task—increased stress, for example. The cognitive system is not directly measured by a particular device but must instead by evaluated according to a combination of such measurements.

The output analysis preferably includes a set of instructions for a feedback integrator, which in turn provides specific instructions to each feedback output device. The abstraction provided by the feedback integrator also makes it easy to add different types of feedback devices, as well as providing context awareness as described above and also enabling certain feedback devices to be ignored if necessary, to reduce computational complexity.

A non-limiting exemplary description of processing biosignal data is provided in U.S. patent application Ser. No. 15/875,227, filed on Jan. 19, 2018 (“SYSTEMS, METHODS, DEVICES AND APPARATUSES FOR DETECTING FACIAL EXPRESSION”), owned in common with the present application and hereby incorporated by reference as if fully set forth herein.

As shown, controller 108 preferably comprises a plurality of biosignal data inputs 114, of which two are shown as inputs 114A and 114B for the purpose of illustration only and without any intention of being limiting. Biosignal data inputs 114A and 114B are preferably integrated by a biosignal data integrator 150, which for example may combine the inputs to a single vector space (such as Hilbert space, for example), and/or may combine biosignal data for a specific purpose, such as for motion tracking. Optionally, motion tracking is provided by a motion integrator 152. Motion integrator 152 may receive separate motion inputs 154A and 154B, for example, from an inertial sensor, a camera, a depth sensor, other motion sensing devices, or some combination thereof.

Optionally, these various signals and/or integrations are analyzed by an analysis engine 156. The analysis results are then optionally provided to a feedback integrator 158, for providing feedback to the patient. The feedback may be provided for example as feedback 160A-B.

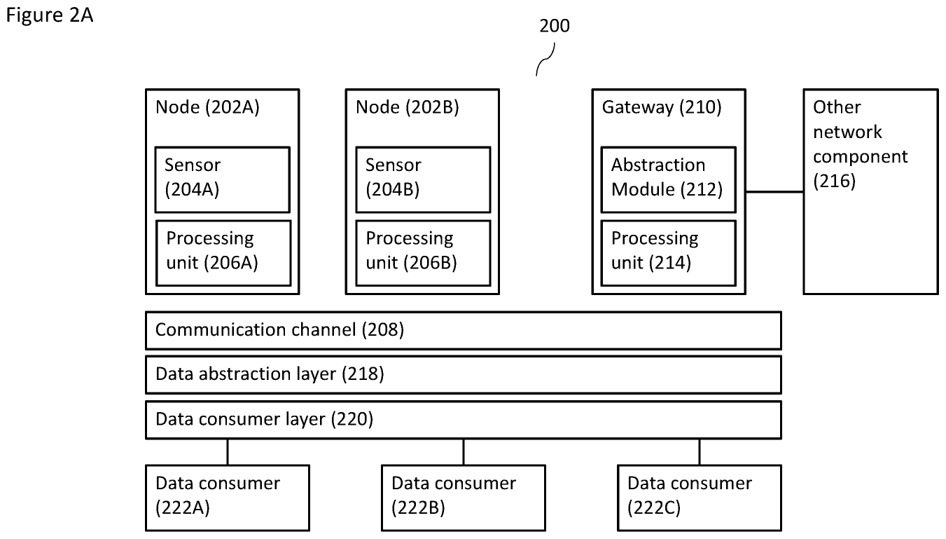

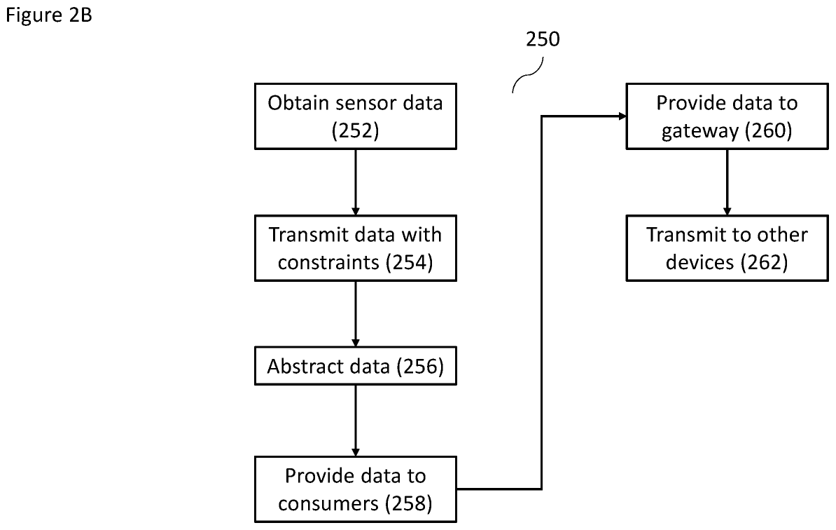

FIG. 2A shows a schematic of a non-limiting, exemplary system for receiving data from a plurality of sensors and distributing it to a plurality of data consumers in accordance with embodiments. As shown, a system 200 features a plurality of network nodes 202A and 202B, of which two are shown for the purpose of illustration only and without any intention of being limiting. Each node 202 features a sensor 204 and a processing unit 206.

Each sensor 204 may comprise any type of suitable sensor, for example and without limitation, EEG, EKG, inertial sensors, IMU, EMG, optical, depth and so forth.

Each processing unit 206 may comprise any suitable type of processor. As used herein, the term processor generally refers to a device or combination of devices having circuitry used for implementing the communication and/or logic functions of a particular system. For example, a processor may include a digital signal processor device, a microprocessor device, and various analog-to-digital converters, digital-to-analog converters, and other support circuits and/or combinations of the foregoing. Control and signal processing functions of the system are allocated between these processing devices according to their respective capabilities. The processor may further include functionality to operate one or more software programs based on computer-executable program code thereof, which may be stored in a memory (not shown). The memory may be in communication with and/or a part of processing unit 206. As the phrase is used herein, the processor may be “configured to” perform a certain function in a variety of ways, including, for example, by having one or more general-purpose circuits perform the function by executing particular computer-executable program code embodied in computer-readable medium, and/or by having one or more application-specific circuits perform the function. As a non-limiting example, processing unit 206 may be implemented as a FPGA (Field Programmable Gate Array).

Each processing unit 206 may perform various types of preprocessing of the signals obtained from its respective sensor 204. Next, the signals are passed to a communication channel 208. Communication channel 208 may comprise any suitable type of communication channel, whether implemented in hardware, software or a combination thereof. For example, communication channel may comprise a hardware (chip) bus for communication between components. Communication channel may also comprise a system of hardware components that are in communication through software abstraction, such as for a computer network for example. Communication channel 208 is preferably implemented deterministically with nodes 202, such that each node is assigned a specific window or other mechanism for transmitting data, optionally with other suitable constraints. For example, such constraints could include order and/or priority of transmission, with certain processing units 206 receiving priority over other such processing units 206.

The deterministic architecture enables nodes 202 to share communication channel 208, optionally without the need for an additional master or controlling node. At the time of implementation of this portion of system 200, the number and types of nodes 202 may be determined. The window or other mechanism through which nodes 202 share communication channel 208 may also be determined at this time. Sharing could be determined according to a suitable network protocol, for example according to a time or frequency based window, as set up for example before run time. Preferably communication channel 208 is part of a deterministic communication system that is set up with nodes 202 and the other components of system 200.

To enable other types of devices to communicate with communication channel 208, preferably a gateway 210 receives data and abstracts such data through an abstraction module 212. The instructions of abstraction module 212 are preferably executed by a processing unit 214. Other devices that are not subjected to the same constraints as nodes 202 may then access the data, through communication with gateway 210, such as for example other network component 216.

Communication channel 208 is preferably in communication with a data abstraction layer 218, which abstracts the data for consumption by other devices and/or software applications. Data abstraction layer 218 enables applications to receive the data which are not subject to the same constraints as nodes 202. Preferably, a data consumer layer 220 transmits the data to a plurality of data consumers 222A-222C, of which three are shown for the purpose of illustration only and without any intention of being limiting. Data consumers 222A-C may, for example, be software applications operated by a computational device (not shown). Such a computational device does not need to be a specialty computational device.

FIG. 2B shows a flow of a non-limiting, exemplary method for providing data to a plurality of networked notes through a communication channel in accordance with embodiments. As shown in a method 250, the process preferably starts by obtaining sensor data at 252. The data is then transmitted according to one or more constraints 254, which may be imposed, for example, according to the communication channel and/or according to the system architecture, as described, for example, with regard to the system of FIG. 2A.

Next, the data is preferably abstracted in 256, to permit sharing of the data beyond the system, which is limited by one or more constraints. The abstraction process also preferably enables a plurality of sensor devices to be connected and to provide data without requiring the receivers of such data to be aware of the exact sensor device type and communication requirements of the sensor device, the data provided by the sensor device, both, or some other communication requirement imposed by the system.

Next, data is provided to one or more data consumers 258, which may, for example, comprise one or more software applications that receive the data for further analysis and/or processing. The data is also optionally provided to a gateway in 260, for transmission to devices beyond the constrained communication network. The gateway is then able to transmit the data to these other devices in 262.

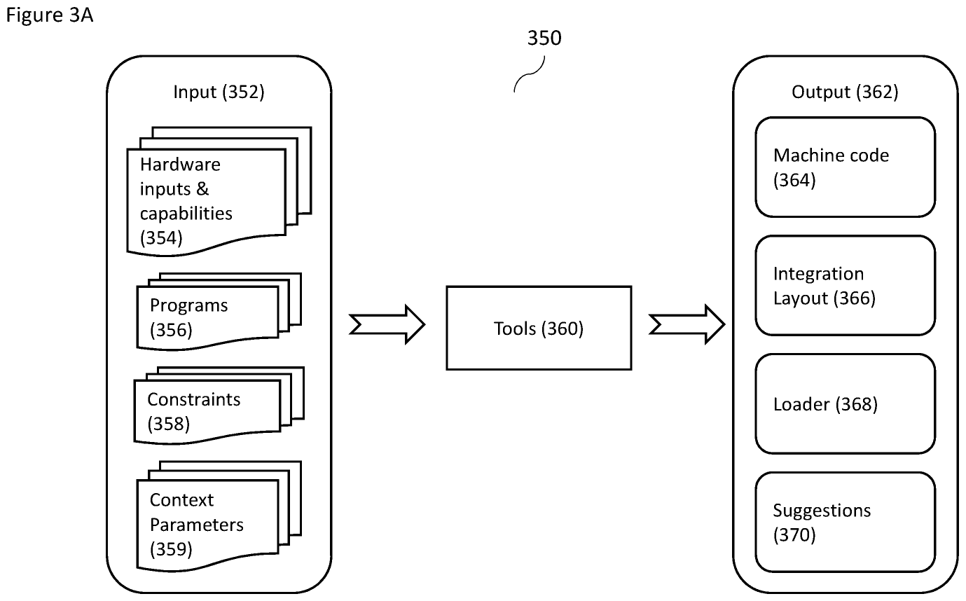

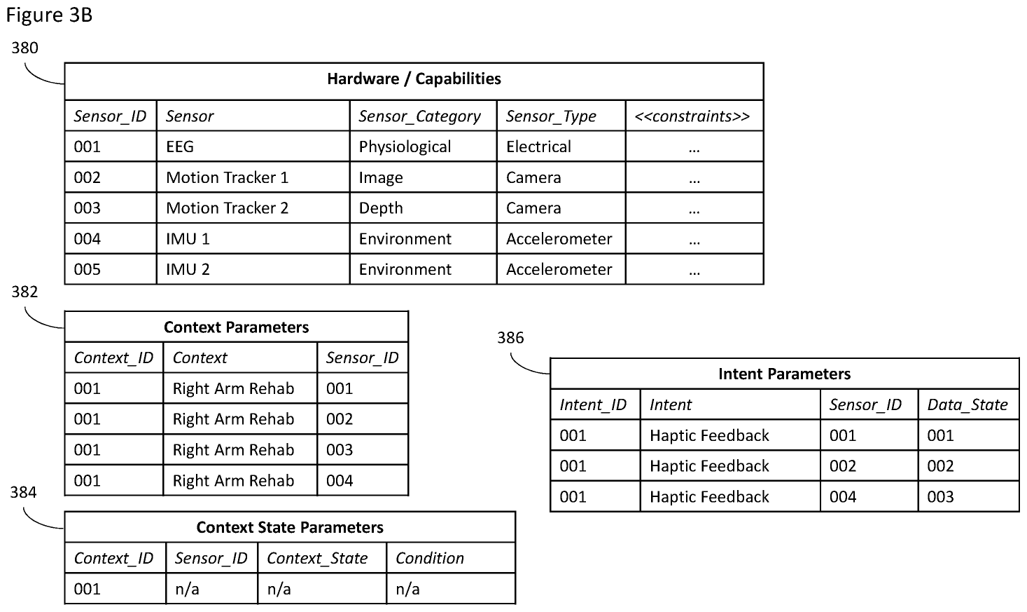

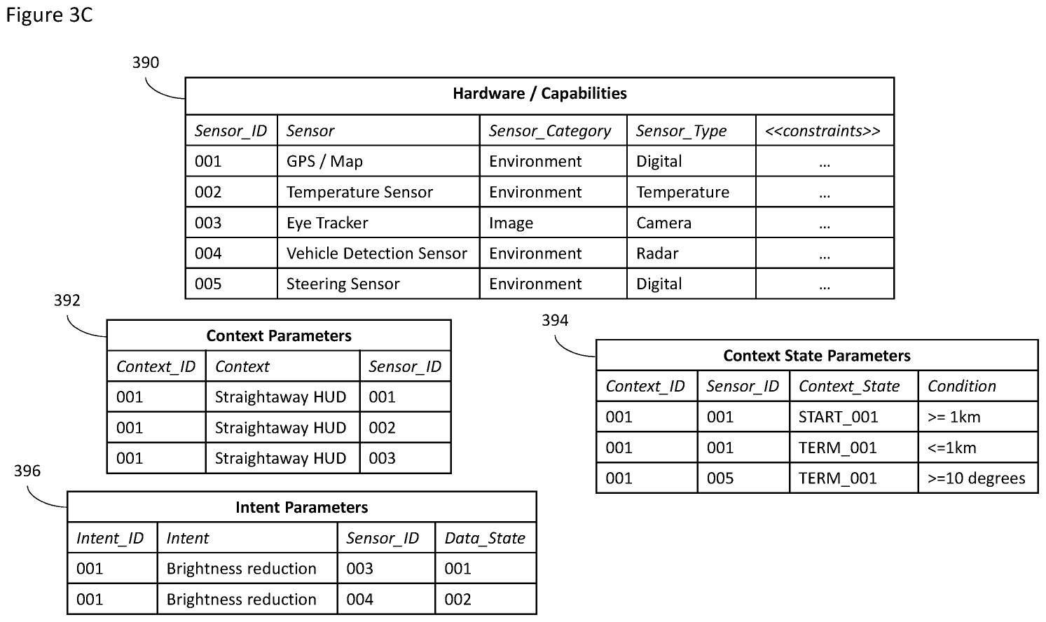

FIG. 3A shows a schematic of a non-limiting, exemplary operational control platform for supporting the above implementation in accordance with embodiments. The above is preferably implemented through an operational control platform, as described herein, that, among other things:
Handles device abstraction (input or output)
Provides messaging and buffer capabilities
Supports state machine control of the various devices
Supports prioritization of controller resources, such as on-chip resources if the system is configured to include a chip

The operational control platform, an optional controller, and external devices together may optionally form a therapeutic platform. Such a platform preferably provides context aware rehabilitation for the patient. In addition, the platform preferably provides reports that are comprehensible to humans and that can be made compatible with future AI (artificial intelligence) diagnostic systems. The operational control platform, controller, external devices, or some combination thereof can be use in environments or for uses other than medical or therapeutic environments or uses. For example, such a system can be used in transportation settings, wellness and athletic settings, and the like. Environments in which sensor data can be used to generate feedback can benefit from the advantages provided by preferred embodiments of the present invention.

The components of the system and their operating requirements are preferably considered in two separate situations. In a first situation, these factors are analyzed by a developer, for example, to determine how to create programs for the system, and/or whether to add and/or change one or more hardware components. In a second situation, these factors are analyzed at the time of system initiation or loading, to analyze the system architecture and to load one or more programs for operation over the system.

Turning now to FIG. 3A, there is provided a schematic of a non-limiting, exemplary implementation of a system for determining the architecture of a platform for supporting data collection and provisioning in accordance with embodiments, and for supporting the boot or initiation of functioning of such a platform. The platform may be described as a Heterogenous Architecture Toolbox (HAT), which is able to integrate communications and control of a plurality of heterogenous hardware and software components. The HAT preferably supports context awareness of the components in the system, in terms of the presence of the various hardware components and also the type of environment or situation in which the data is being collected and processed. The HAT is also preferably able to support real time collection of data and overall real time operation of the hardware components of the platform. By “real time” it is meant that operation of the platform meets the necessary constraints on its behavior according to the timing specified, such that an architecture can be created that allows for real-time operation based on the given constraints of the system's devices.

As shown in a system 350, there is provided input 352, a plurality of tools 360 and an output 362. Input 352 relates to information provided to a developer as well as to the product of development that is to be made ready for execution, for example through a compiler driven process. As shown for input 352, preferably hardware inputs and capabilities 354 are provided. These inputs and capabilities 354 are static, such that they are part of the architecture of the platform determined according to system 350. The architecture is preferably static to be able to provide a bound time delay for the services provided. This means that each part of the platform architecture behaves in a way so that the time between the occurrence of an event and the processing of that event, called the latency or lag, is guaranteed to be both short and bounded. Broadly speaking, this means that the behavior of the hardware is predictable.

For example the communication channel in FIG. 2 may have its behavior determined by system 350 such that it induces a predetermined delay, which may be a maximum possible delay, on the information it carries; the maximum is certain without a doubt, and preferably reasonably small. Therefore the resultant platform preferably behaves with predictable reliability, without requiring over-provisioning of the hardware. As another example, if an exceptional event occurs (e.g., interruption, event, computed metric, and the like), system 350 preferably specifies under which conditions the event may occur, and reserves hardware resources to handle such an event without affecting the whole system.

Such static features of the platform determined by system 350 provide a number of advantages. Without wishing to be limited by a closed list, static features mean that upon boot up or initiation, the platform whose architecture is determined by system 350 may operate in real time, for example to gather sensor data in real time.

In addition, one or more programs 356 are provided, which may have any suitable function, including with regard to gathering data from the sensors, consuming data, or providing data. Preferably one or more constraints 358 are also provided to determine the requirements for the hardware components of the system, including the tools. Constraints 358 are evaluated against inputs and capabilities 354, to determine whether the capabilities have been properly accounted for. Furthermore, constraints 358 are evaluated against the inputs to determine whether all hardware components have been considered for system 350. Such evaluation may lead to a determination that one or more hardware components, and/or one or more characteristics of such hardware components, have not been properly accounted for. In such a situation, preferably the loading process stops until these problem(s) have been rectified.

Inputs 352 can also include context parameters 359. Context should be understood to mean an environment or set of conditions in which data is to be collected from an array of sensors and other devices to be fused for creating feedback or adjusting parameters that determine whether and/or how data is collected or fused. For example, in a medical setting, a context could be a particular type of rehabilitation of the right arm of a stroke patient. In that case, referring to FIG. 3B, a context parameters profile 382 could include parameters that indicate which of the hardware inputs are relevant for the context such as EEG, motion tracking devices, and an IMU, for example. Other examples could include components of a sensing system such as motion tracking markers where, for right arm rehabilitation, perhaps only right arm motion tracking markers are relevant. Those of skill in the art can appreciate the different types of sensors that could be used in such rehabilitation contexts. In other words, the parameters could indicate the sensors in a neurorehabilitation system from which physiological or other data are processed, fused together, and the like to generate feedback and the like. The overall rehabilitation system can include a larger universe of sensors/devices, for example, than is shown in hardware parameters 380, that are relevant for other contexts for which the rehabilitation system is used. Hardware parameters 380 can include hardware devices that are or may be used in the system. Preferrably, it includes items for all the actual hardware devices to be used in the various relevant contexts. Hardware parameters 380 can include basic information about the hardware devices such as an identifier, name, type, manufacturer, and the like. Additionally, hardware parameters 380 preferrably includes constraint information about the hardware devices that are material to tools 360 and output 362. Such constraint information can include a constraint type and value. Examples of constraints include but are not limited to operating frequency, communication frequency, communication type (e.g., wired/wireless), communication signal strength, power consumption, power capability, memory buffer capacity, memory capacity, and the like. In particular, operating frequency and communication frequency are significant to ensure real-time fusion of multiple sensors and feedback generation.

Parameters can further include intent parameters 386 that can be used to indicate how feedback is determined from the sensor/device data. Keeping with the same example, a haptic device may be used in right arm rehabilitation. Where EEG, motion tracking, and IMU data indicate a certain state by virtue of a combination, or fusion, of their data, haptic feedback can be presented. Parameters can further include context state parameters (further described in connection with FIG. 3C) which may or may not be relevant in some contexts. In a rehabilitation context, it is normally desirable to present feedback to the patient and thus context state parameters may be less common.

Some preferred embodiments could be used in non-medical settings, such as driving, for example. In that case, a context could be a driving condition such as driving in a straight path. Parameters for such a context are illustrated in FIG. 3C in accordance with some embodiments. In this example, a system can provide heads up display elements based on physiological, image, and other sensor data under certain driving conditions when a vehicle is traveling in a straight path. Here, context can be straightaway HUD (heads up display), such that while driving along a straight-line path the HUD is presented to the driver. If the vehicle is not proceeding along a straight-line path, optionally the HUD is not shown. Hardware input parameters 380 can include an eye tracking image sensor, a vehicle detection sensor, an environment sensor (e.g., exterior temperature sensor), GPS/map system, and the like, along with the capabilities and constraints of the sensors and other of these devices. The parameters could be organized into a first group and a second group. The first parameter group, the context state parameters 394, could indicate the sensors and/or devices used to determine when the context is active (i.e., when the vehicle is traveling in a straight line). Here, the first group would include sensor parameter of GPS/map. For example, if the GPS/map data shows that a vehicle is traveling a sufficiently straight path (e.g., >=1 km of straightaway) and may continue to do so, the context would be active (i.e., the vehicle is in a state of straightaway travel). Where the straightaway path is detected from the GPS/Map to be under a threshold (e.g., 1 km) or the steering sensor indicates a turn beyond a threshold (e.g., 10 degrees), the state is inactive, or should be terminated. The second parameter group, the intent parameters 396, could indicate the sensors and/or devices from which data would be used to generate real-time feedback. In one example, this second parameter group could include eye tracking sensor and vehicle detection sensor. Here, the system could be used to alter the appearance of the HUD or present other display elements of the HUD if it is determined that the driver's eyes wander from road, if they become droopy, when other vehicles are present but not visible, and the like, or some combination thereof.

FIGS. 3B and 3C present examples of organization parameters used to define and/or describe contexts and the conditions under which feedback is provided and/or how feedback is provided. It should be understood that FIGS. 3B and 3C are non-limiting and such parameters or information can be organized in other ways in accordance with preferred embodiments.

Context parameters thus enable the addition or exchange of new devices or sensors with different capabilities and/or constraints or the addition of new contexts without the addition of new hardware, including sensors, other input devices, feedback/output devices, or any hardware specific to the processing of the sensor/device data.

It should be understood that embodiments can be used in other settings and the settings and contexts described above are non-limiting. For example, air transportation is another setting in which data from multiple, disparate sensors and sensor types, to the exclusion of physiological data, could be fused to generate real-time feedback or other feedback.

Turning back to FIG. 3A, inputs 352 are provided to a plurality of tools 360. Such tools may include but are not limited to one or more of memory mapping, ordering, pipelining, task mapping, scheduling, optimization and output generation. For example, tools 360 may be able to compile programs 356, optionally according to context awareness for other components of the system.

Tools 360 preferably support integration of, and communication between, the plurality of hardware components, preferably including a plurality of sensors. As previously noted, preferably sensors comprise and/or are in communication with a processor for performing at least preprocessing. In such an implementation, tools 360 preferably support bidirectional communication between the processors, as well as from the sensor (directly or through a processor) to a central processing unit of some type. For example, this would support combining data from two sensors to generate a derivate measure at the central processing unit and/or by another processor in the system. This more flexible architecture could also reduce bandwidth required in the communication channel. Such architecture also increases the constraints on the system, to enable such multi-directional communication.

Operations of tools 360 leads to the creation of one or more outputs 362, including, for example, the generation of machine code 364 according to the operation of a compiler. The resulting output would contain the machine code 364 for all the different microprocessors, a map with locations (absolute or relative) of the sensors, processors, their communication inputs and outputs; and the occupancy of each communication channels, CPUs, and memories available. This map is described below as an integration layout 366. Tools 360 would generate such an integration layout 366 by solving an optimization problem with constraints. One approach could be to use Monte Carlo methods like simulated annealing.

The combination of constraints 358, inputs and capabilities 354, and programs 356, preferably results in the production of integration layout 366, through plan scheduling and mapping of tasks. Integration layout 366 may for example relate to a software blueprint or a diagram layout. A software blueprint preferably shows all relevant logic, localized in a suitable format. As a non-limiting example, integration layout 366 may be implemented using a CDL (Concurrent Description Language) which separates the macroscopic logic (communication, synchronization and arbitration) of system 350 from complex multi-threaded and/or multi-process applications into a single contiguous visual representation. The prescriptive nature of this description means that it can be machine-translated into an executable framework that may be tested for structural integrity (e.g., detection of race conditions, deadlocks, etc.) before the microscopic logic is available. In this case, integration layout 366 preferably expresses both the deterministic architecture and its associated processes.

Integration layout 366 may also specify whether system 350 is to feature one or more redundancies, whether in the presence of multiple sensors providing the same or similar data, or in terms of multiple nodes for analyzing the data and/or otherwise executing programs. Such “N-version programming” may be used to avoid partial or complete system failure.

Programs 356 are also preferably compiled into machine code by a suitable compiler in tools 360, to form machine code 364. Optionally, upon compilation, programs 356 are combined with a wrapper. A wrapper acts as an interface between a module and firmware. It preferably verifies both the inputs and the outputs of the wrapped software. Such wrappers may act to “catch” errors before they disrupt operation of system 350, for example by filtering any incorrect value that passes through it. For such an implementation, the wrapper may be able to prevent incorrect values from being given to a program and also preferably to perform acceptance tests on the output variables. The necessity for a wrapper and/or the actual wrapper may be specified by integration layout 366. Compilers are well known in the art and any suitable compiler can be used.

Another exemplary output 362 is loader 368. Hardware inputs and capabilities 354, programs 356, and integration layout 366 are preferably combined to generate loader 368. Loader 368 may be a boot loader and/or a firmware loader. For example, in the paper by Anton, et al., “Firmware and bootloader” (March 2012, Rose website of Paris Telecom (https://rose.telecom-paristech.fr)), the authors discuss embedded bootloaders and firmware. In particular, general constraints for embedded systems, including but not limited to time-critical computing environment, low-power, fault tolerance or low-cost constraints are considered. One or more of these constraints are also preferably considered with regard to implementation of loader 368.

Loader 368 also preferably determines whether one or more wrappers, associated with one or more programs 356, have been successfully compiled, combined in the final output machine code 364, or both. Loader 368 also preferably considers whether N-version programming has been implemented, according to information from integration layout 366, and then determines whether necessary components for N-version programming have been successfully loaded. Loader 368 may also be able to support forward and backward recovery, and/or to determine whether sufficient components of system 350 have been successfully loaded to support forward and backward recovery.

Loader 368 also preferably specifies the loading sequence and causes this sequence to be implemented, preferably according to the received information. For example, certain nodes may need to be booted before one or more sensors can be brought online. A non-limiting example of a boot sequence is provided in the above described paper by Anton, et al.

The initiation process preferably results in the generation of a log, for example in the form of suggestions 370. The log preferably includes meta-information generated by the previously described processes. It preferably includes how the layout fits the constraints, compilation warnings, and metadata on the code generated. It preferably also highlights possible bottlenecks.

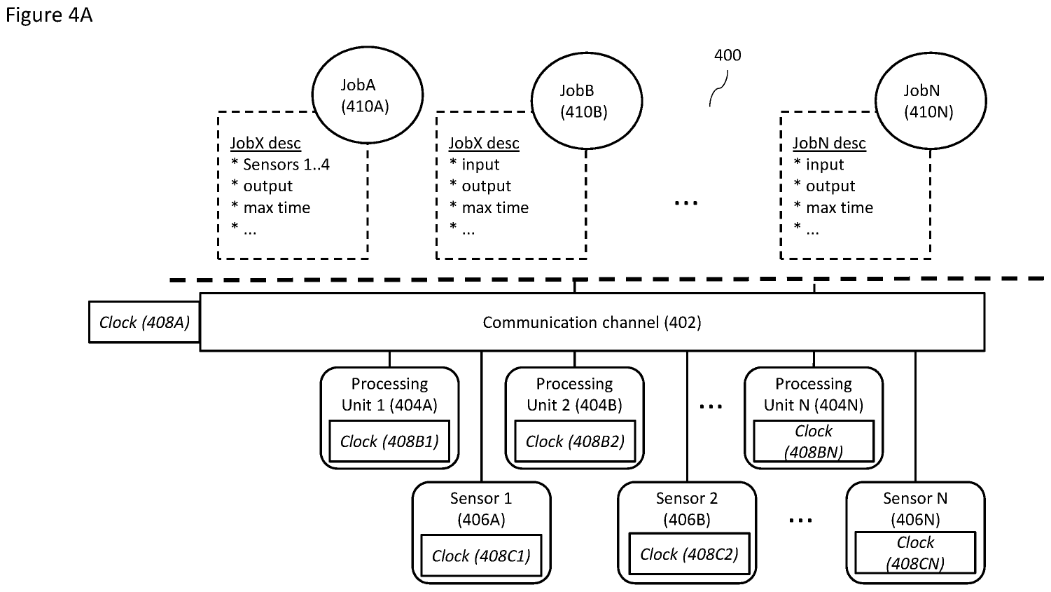

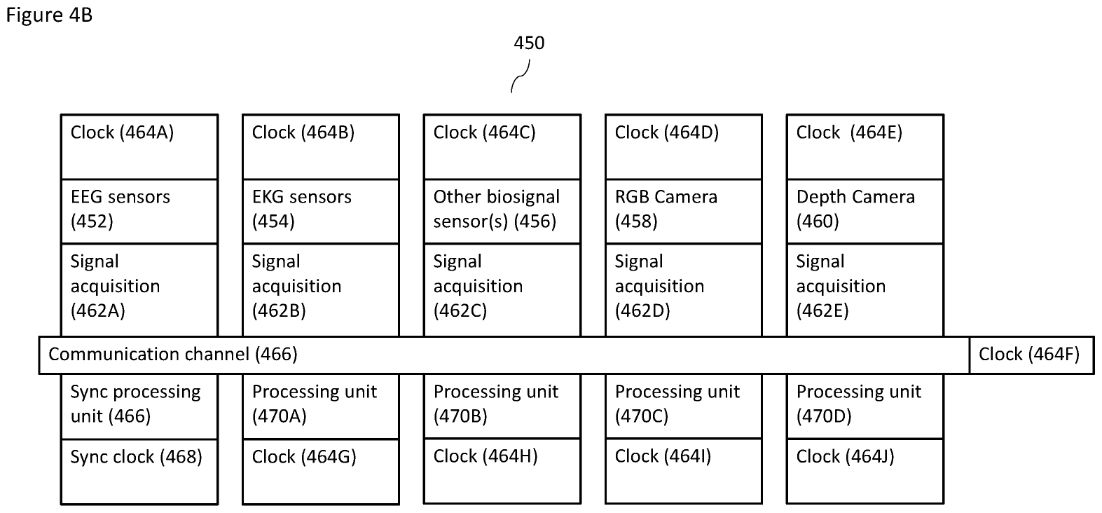

FIGS. 4A and 4B relate to schematics of non-limiting, exemplary systems for providing an operating system according to at least some embodiments of the present invention. As shown, FIG. 4A provides a system 400 for performing a plurality of jobs in real time according to a plurality of constraints, in terms of requirements for the system operation, the plurality of jobs shown above the dotted line. Below the dotted line, system 400 includes a hardware platform, featuring a plurality of sensors 406A-406N and associated processing units 404A-404N, each with one or more separate capabilities and specific I/O (input/output).

Within system 400, a communication channel 402 provides communication for the various components, receiving various jobs 410 (shown as jobs 410A-410N for the purpose of description only and without any intention of being limiting) and then receiving data for such jobs, and optionally, executed job output, from a plurality of processing units 404 (shown as processing units 404A-404N for the purpose of description only and without any intention of being limiting). If communication channel 402 is on a chip, then it may be implemented as a bus for example.

Processing units 404 receive data in real time from a plurality of sensors 406 (shown as sensors 406A-406N for the purpose of description only and without any intention of being limiting). Sensors 406 may be any type of suitable sensor, including without limitation sensors for biosignals to obtain biosignal data, such as EEG, EKG, EMG, GVR and the like; as well as sensors for measuring human movement and/or interaction, such as inertial sensors, IMUs, optical (RGB) cameras, depth sensors and the like; and/or an actuator or position sensor (hall sensor, motor encoder and the like). Sensors 406, processing units 404, and communication channel 402 are preferably synchronized through a plurality of clocks as shown. Clock 408A is a communication channel clock for timing communication channel signals; clocks 408B1-408BN are processor clocks for synchronizing processing units 404; and clocks 408C1-408CN are sensor clocks for synchronizing sensors 406. In some embodiments, communication channel 402, processing units 404, and sensors 408 each have their own clock 408A, 408B1-N, 408C1-N. In other embodiments, it should be understood that some devices may share a clock.

It should be understood that clocks 408A, 408B1-N, 408C1-N represent the time relative to a sensor or processing unit and may not therefore be absolutely synchronized unless they derive from a common clock signal. However, it is possible to relate each individual clock signal relatively to a common time reference provided and maintained by one or several processing units 404 or other nodes (not shown). There exist approaches to maintain the correspondence of an individual clock signal with respect to the common time reference, a non-limiting example of which is the NTP protocol for maintaining time of computers. Data acquired or processed by a sensor 406 or processing unit 404 are paced by the clock of each individual device or node (not shown), but can be tagged with respect to the common time reference. It is possible to keep track the processing delay of a combined modalities (say from sensors 406 with different clocks 408). Also data from different clocks 408 can be compared through resampling after interpolation. Depending on the context, such operation can be costly and thus may be limited to what is required. A scheduling tool preferably take such constraints into account.

If the requirements for transmission and/or receipt of data through communication channel 402 is set in advance according to a deterministic architecture, then optionally no synchronization controller or master is required. Communication channel 402 may comprise a hardware bus or a combination of hardware and software components, as described previously with regard to FIGS. 2A and 2B.

FIG. 4B shows a schematic of a non-limiting, exemplary system for receiving data through a communication channel, for consumption by communicating network nodes in accordance with embodiments. In a system 450, a communication channel 466 preferably communicates with a plurality of components. Communication channel 466 may comprise a hardware bus or a combination of hardware and software components, as described previously. A plurality of sensors provide information through communication channel 466, including without limitation an EEG sensor 452, an EKG sensor 454, optionally other biosensor(s) 456, an RGB camera 458 and a depth camera 460.

Data from each of these sensors is then acquired and preprocessed for distribution through communication channel 466 by a signal acquisition component 462 (shown as signal acquisition components 462A-462E for the purpose of description only and without any intention of being limiting). Signal acquisition component 462 preferably abstracts the data and then provides it according to the constraints required by communication channel 466 specifically and/or system 450 generally.

The data may then be consumed by one or more of a plurality of processing units 470 (shown as processing units 470A-470D for the purpose of description only and without any intention of being limiting). Processing units 470 may for example analyze the data to determine a result, including without limitation, the biological or emotional state of the user, a movement of the user, a diagnostic state of the user, feedback for the user, and the like.

In this non-limiting example, a sync processing unit 466 controls synchronization of the various components of system 450. Each component of the system is preferably synchronized according to a clock 464 (shown as clock 464A-464E for the purpose of description only and without any intention of being limiting), controlled through a sync clock 468 as shown.

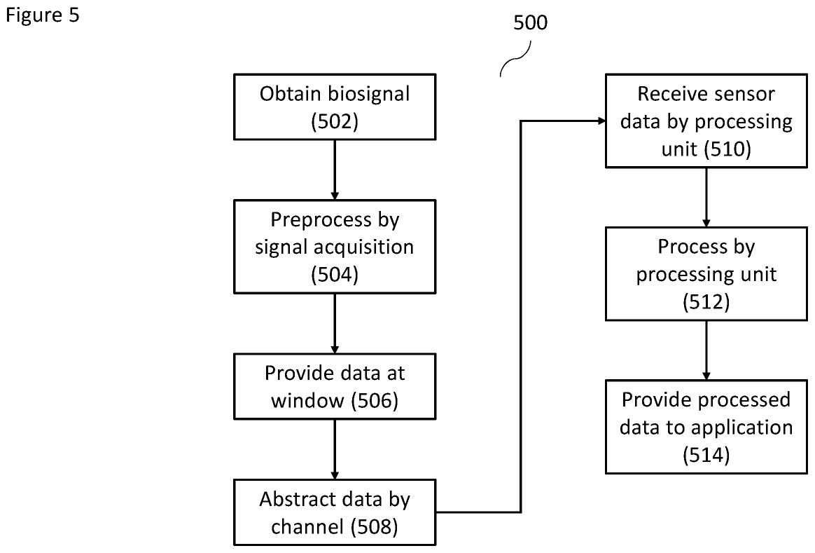

FIG. 5 relates to a flow of a non-limiting, exemplary method for obtaining biosignal data and providing the data to one or more consuming applications, such as software applications for example, in accordance with embodiments. The term “biosignal” as used herein may relate to any type of signal obtained from, or according to, activity of a user. As shown, a method 500 begins with obtaining the biosignal data at 502. Preferably the biosignal data is preprocessed by a signal acquisition component at 504, to support distribution throughout a network of nodes and/or consuming applications. The data is provided for distribution at an appropriate time and/or frequency and/or channel window at 506. For example, if the system for such data distribution has a deterministic architecture, such a window would provide constraints according to when and/or how the data is distributed.

Next, the data is preferably abstracted by channel at 508, to enable higher level consuming applications to receive the data, without needing to be aware of the lower level hardware and/or other device constraints. For example, such abstraction may be supported in the form of a device driver, for example for the sensor and/or for a device comprising the sensor.

At 510, the processing unit receives the sensor data. The processing unit then processes the sensor data at 512. The processed data may then be provided to a consuming application at 514, such as a software application for example. Alternatively, 512 and 514 may be combined to a single process.

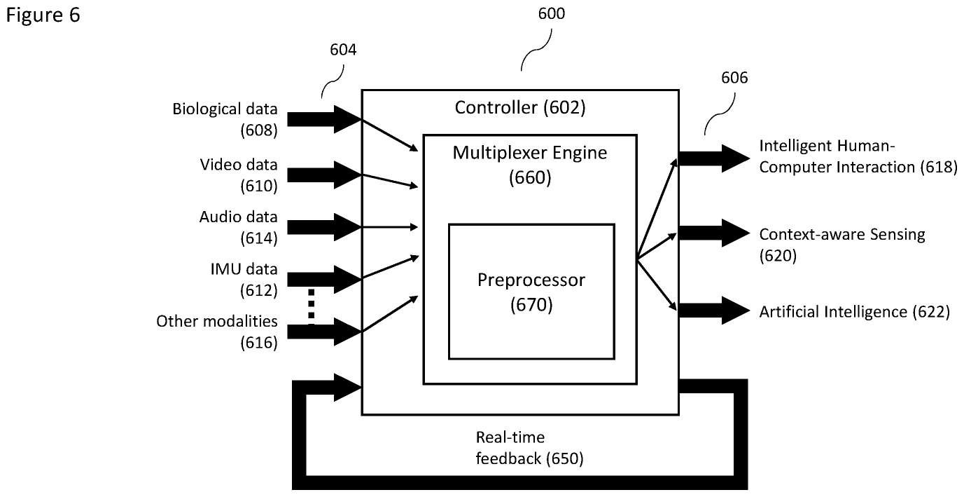

FIG. 6 relates to a schematic of an exemplary implementation of the controller in a functional system in accordance with embodiments. A functional system 600 preferably features a controller 602. Controller 602 preferably supports context awareness for obtaining and analyzing different types of data, provided as a variety of data inputs 604. Such context awareness supports analysis of data from different sensing modalities relative to each other. For example such analysis may result in the merger or fusion of processed data from different modalities. Processing inside controller 602 preferably supports the provision of output 606 as a combination of modalities that are related to each other. Depending upon the context in which data inputs 604 are obtained, analysis may include for example SLAM (simultaneous localization and mapping), video feedback with context, help in localization of sensors and so forth.

Data inputs 604 preferably include but are not limited to biological data 608 which may for example comprise biosignal data (e.g., EEG, EMG, ECG, and the like); video or image data 610 (e.g., Depth, Stereo, infrared, and the like); IMU (inertial measurement unit) data 612, which can include, without limitation, data from one or more gyroscopes, one or more accelerometers, one or more magnetometers, and the like; audio data 614; and optionally other modalities 616, such as inputs from robotic devices and the like.

The controller 602 preferably operates on these data inputs 604 to provide a plurality of data outputs 606. In some embodiments, controller 602 includes a multiplexer engine 660 that operates to relate relevant data from inputs 604 together based on information about the context of the application, for example, as discussed in connection with FIGS. 3A-3C. Multiplexer engine 660 can include a preprocessor 670 that synchronizes the relevant data from inputs 604 according to clock signals, for example in accordance with FIG. 4 described above. Non-limiting examples of such outputs 606 include Intelligent Human-Computer Interaction (618), context-aware sensing (620), and artificial intelligence analysis (622).

Context-aware sensing 620 preferably relates to awareness of the various sensors in the system, providing the previously described types of data 604, and the environment or situation in which they are operating. As a non-limiting example of such context awareness, interacting with a machine using a Brain Computer Interface (BCI) relies on the recognition of cognitive processes based on their correlated brain activity which is most of time the Electroencephalography (EEG). One of the most popular ways to implement such systems is to exploit Event Related Potential (ERP) or evoked synchronization of desynchronization such as automatic responses of the brain to external stimuli (G. Pfurtscheller, C. Neuper, C. Guger, W. Harkam, H. Ramoser, A. Schlögl, B. Obermaier, and M. Pregenzer. Current trends in Graz brain-computer interface (BCI) research. IEEE Transactions on Rehabilitation Engineering, 8:216-219, 2000). Such ERP is Error-related negativity (ERN) which is electrical activity in the brain as measured through electroencephalography (EEG) and time-locked to an external event (e.g., presentation of a visual stimulus) or a response (e.g. an error of commission). It typically peaks from 80-150 milliseconds (ms) after the erroneous response begins. Due to their time-locked nature evoked responses are usually well distinguished from background activity and easily picked out but require the subject being “synchronized to external machinery” (i.e. system-paced).

The issue for interpreting those brain activities is the lack of context. If a vision system is monitoring the scene in synchronization with the monitoring of the EEG, the interpretation of the visual cues provides the context needed to interpret the brain signal, hence could be used to indicate that the subject whose EEG is monitored has realized he just made a mistake. Alternatively, if the vision interprets the interaction of the subject with an external agent (a device or a human), the brain activity synchronized with the vision can help to realize that the external agent has done an action the subject perceived as erroneous.

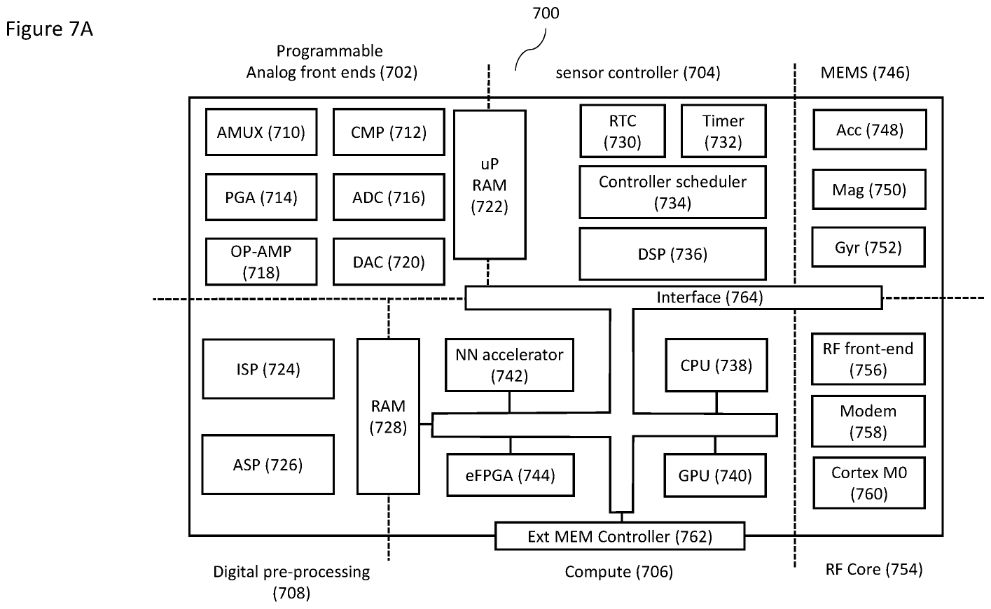

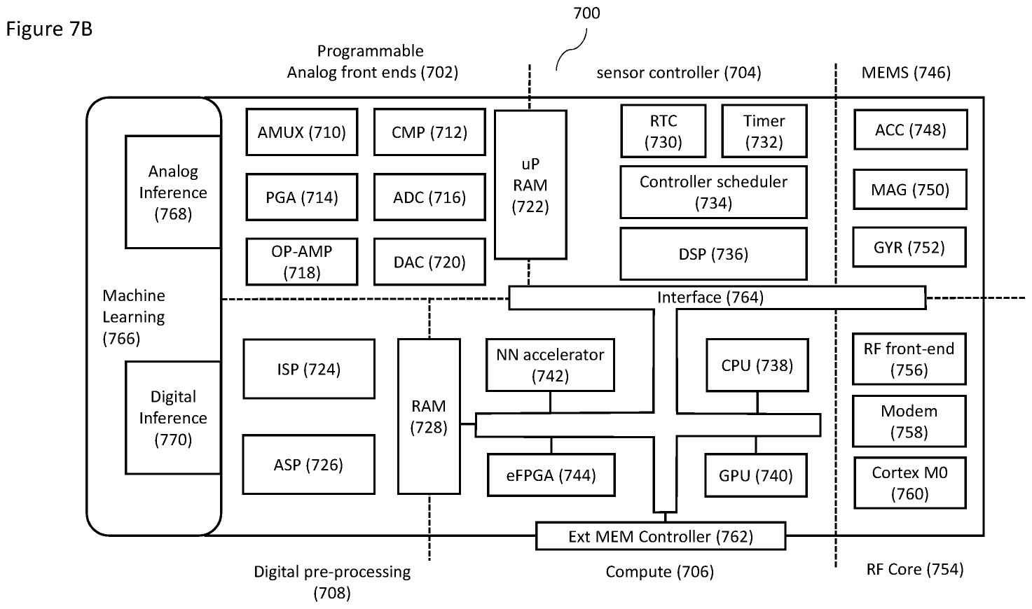

Artificial intelligence analysis 622 may for example relate to the machine learning described with regard to FIG. 7B below, which supports edge preprocessing of the signals from various sensors, as well as analysis of such signals.

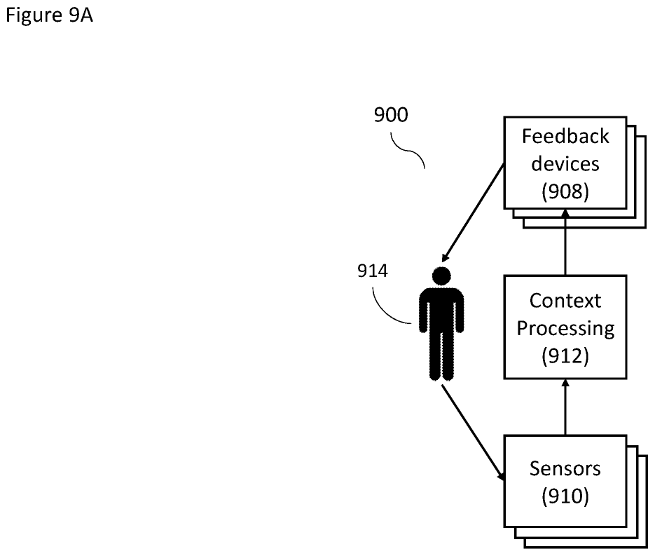

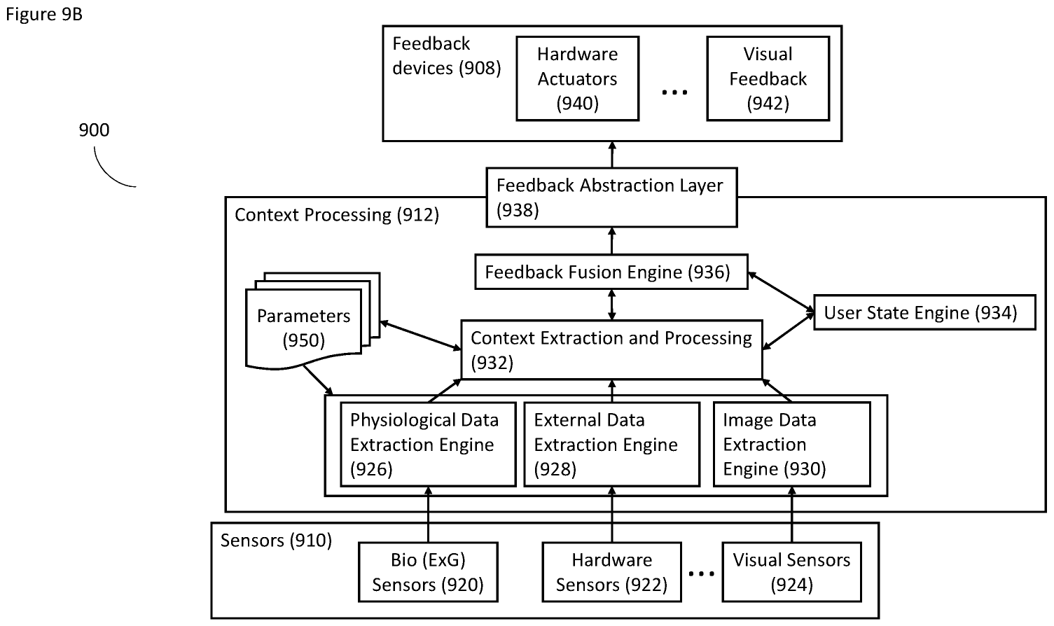

Preferably outputs 606 then act as further feedback to controller 602 in a feedback loop 650. In some embodiments, outputs 606 are used to determine feedback, for example, in accordance with FIGS. 3A-3C as described above and FIG. 9 described below.

As shown, the controller may be configured as a single device or unit, or as a plurality of devices or units.

FIG. 7A relates to a schematic of an exemplary implementation of the controller as a chip in accordance with embodiments. Optionally, the device may be configured as any type of chip as described herein, including without limitation a three dimensional chip, an SoC (system on chip), an analog front end (AFE) configuration, an FPGA (field-programmable gate array) and so forth. A non-limiting exemplary description of a suitable FPGA is provided in U.S. patent application Ser. No. 15/891,235, filed on 7 Feb. 2018 (“SYSTEMS, METHODS AND APPARATUSES FOR STEREO VISION”), owned in common with the present application and hereby incorporated by reference as if fully set forth herein. A non-limiting, exemplary description of a suitable AFE configuration as a SoC (system on chip) is provided in the article by Kim and Ko (“Reconfigurable Multiparameter Biosignal Acquisition SoC for Low Power Wearable Platform”, Sensors (Basel). 2016 December; 16(12): 2002).

As shown, when configured as a chip 700, the controller may feature a plurality of Programmable Analog front ends (AFE) 702, a sensor controller 704, a computational core 706 and a digital pre-processing core 708.

Each AFE 702 preferably features an AMUX (analog multiplexer) 710, a comparator (CMP) circuit 712 for comparing two analog input voltages, a PGA (programmable-gain amplifier) 714, an analog to-digital converter (ADC) 716, an operational amplifier (OP-AMP) 718, a digital to-analog converter (DAC) 720, and a memory, such as RAM 722, for example.

Optionally the AFE may be configured as for the Healthcare Analog Front End (AFE) of NXP (https://www.nxp.com/docs/en/fact-sheet/HCAFEREFPLATES.pdf).

The data signals, such as biosignal data, video data, audio and so forth, are each preferably received by a suitable AFE. The data is then transferred in an appropriate format to the digital pre-processing core 708. The digital pre-processing core 708 preferably features an image data preprocessing core 724 and an audio data preprocessing core 726. Other preprocessing cores, adapted for particular biosignal data, or for a group or category of such biosignal data, may be present (not shown). Preprocessed data is written to a memory, such as RAM 728, which is then accessible to other components of the chip for example through a bus or interface 764 as shown.

Preprocessed data may be accessed by the sensor controller 704 as shown, for feedback to the sensors. The sensor controller preferably controls the sensors, for example, to trigger data transmission and/or activation of the sensor. The sensor controller may feature a real-time clock (RTC) 730 and timer 732 for maintaining correct timing and synchronization. A controller scheduler 734 determines when to trigger or activate the sensors. A DSP 736, which is preferably an ultra-low power DSP, manages the functions of the sensor controller.

The computational core 706 preferably accesses the preprocessed data to perform various calculations and/or analyses. The computational core 706 preferably includes a CPU 738, a GPU 740, optionally a neural network (NN) accelerator 742 to accelerate the performance of any AI engines, and optionally an FPGA 744 for further extensibility.

The controller 700 also preferably features a microelectromechanical system (MEMS) 746, that more preferably comprises an accelerometer (ACC) 748, magnetometer (MAG) 750 and a gyroscope (GYR) 752. The controller 700 also preferably features an RF (radiofrequency) core 754, comprising an RF front-end 756 as the interface, a modem 758, and a processor 760, such as the Cortex MO 32 bit ARM processor. The controller 700 may also communicate with an external MEMS system through a MEMS interface 762. Optionally all of the components of controller 700 communicate through an interface 764.

FIG. 7B shows a schematic of an optional additional implementation of the chip of FIG. 7, with the addition of edge preprocessing of the data signals. Such edge preprocessing preferably supports data prefiltering to select the most important signals, more preferably in a context aware manner, so that only important signals and data are delivered to the chip for processing. Such edge preprocessing may be accomplished as shown with an analog inference module for the AFE and/or a digital inference module for the digital pre-processing core. Either or both inference modules may be implemented for loading/mapping trained machine learning models as shown with machine learning modules 766.

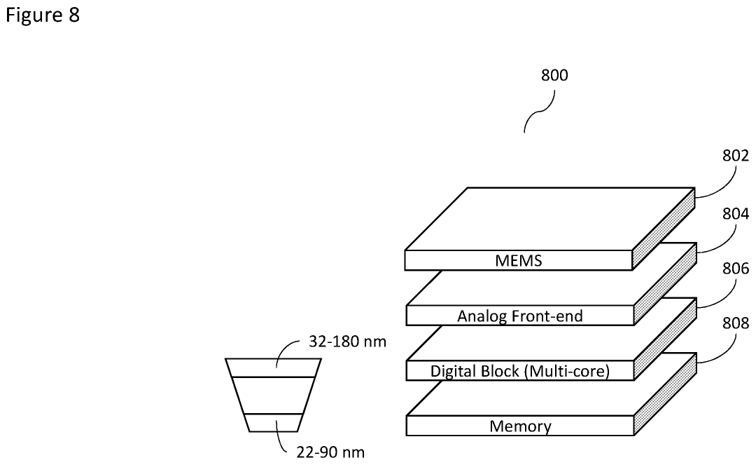

FIG. 8 shows a schematic of an exemplary implementation of the chip as a 3D (three dimensional) chip, shown as a chip 800. Chip 800 features a MEMS 802, an analog front end 804, a digital block (preferably multi-core) 806, and a memory 808. As a non-limiting example, the size and thickness of the components may be implemented according to the scale as shown, although these specifications are not required.

### Suitable Input and Output Devices

Suitable biosignal data input devices (sensors) include but are not limited to:
Heart rate
Galvanic skin response
Blood pressure
EEG
EMG
EKG
Oximeter
   Suitable motion input devices include but are not limited to:
RGB camera
Depth camera
   Suitable output devices include but are not limited to:
Visual display
Audio feedback
Haptic feedback
Text output (for example for a log file)
   Other types of patient information (external to the measurement devices) include but are not limited to:
Medical diagnosis (days post-stroke, stage of recovery etc)
Medical prognosis by therapist, doctor etc
Physical condition (missing a limb, etc)
Psychological condition (reaction to damage/injury, available social/emotional support, etc)
Cognitive state/limitations

In FIG. 9A, a general flow 900 of a feedback loop in accordance with embodiments is illustrated. The feedback loop includes feedback devices 908, sensors 910, context processing 912, and a patient 914. FIG. 9B illustrates a schematic of a portion of the data flow 900 and a non-exhaustive diagram of system components used therein. Featured in data flow 900 are sensors 910 which can include biological sensors 920, hardware sensors 922, visual sensors 924, and the like. Biological sensors can include EEG, EKG, EOG, EMG, and other types of sensing devices that collect physiological data. Hardware sensors include such sensors as IMUs, such as accelerometers, magnetometers, gyroscopes, and the like; environmental sensing devices, such as temperature, pressure, and the like; other types of non-biological or physiological sensing devices or visual sensors; an actuator or position sensor (hall sensor, motor encoder and the like); and/or LIDAR, ultrasound, milimeter wave radar and the like. Visual sensors 924 can include image capture devices, eye tracking devices, depth sensors, other camera-based sensors, and the like.

Sensor data is received by the context processing module 912 in which the feedback loop is realized. Physiological data is received by a physiological data extraction engine 926. Here, context parameters from parameters 950 as described in connection with FIGS. 3A-3C can be used to determine which relevant physiological data is extracted or received. Hardware sensor data is received by an external data extraction engine 928 and image or visual sensor data is received by an image data extraction engine 930 in similar ways according to context parameters. Each of engines 926-930 can include one or more AI inference engines, similar to the inference engine described in connection with FIG. 7B. An AI inference engine can be specific to a type of sensor data and can be trained for a specific context (that is, the relevant neural network can be deployed for a context). Each of engines 926-930 can also include preprocessing that is particular to the relevant types of sensor data extracted such as denoising or other techniques widely known in the art for the relevant types of data received by engines 926-930. Each engine 926-930 preferrably includes preprocessing modes specific to its type of sensor data received.

Relevant, preprocessed data is preferrably received by a context extraction and processing engine 932 where sensor data is fused. Sensor data fusion can be accomplished, for example, using a Kalman filter. Fused data can be sent to feedback fusion engine 936 for the determination of feedback to be generated for the user or the system, depending on the context. Feedback determined can include feedback provided by feedback devices 908 or feedback provided to the context extraction and processing engine 932 in the form of adjustments to parameters 950 or aspects of activities provided by the system. For example, in a rehabilitation context, sensor data can indicate that a user is unable to move a limb to meet the requirements of an activity in a particular context. In that case, feedback fusion engine 936 can provide feedback to context extraction and processing 932 that activities for the context should include others in which the user may meet the requirements or that activity difficulty levels should be reduced. Furthermore, visual sensor data or other sensor data can show that the user intends to move the limb (e.g., eye tracking sensor data showing eyes focused on limb, motion tracking sensor data, IMU sensor data, or force sensor data showing movement within a range of connected joints in the user's anatomy). When context extraction and processing engine 932 fuses this other sensor data with sensor data showing lack of movement in the limb, feedback instructions for a haptic arm can be generated in realtime by feedback fusion engine 936. The instructions can be generated and sent to feedback devices 908 by virtue of the sensor data being received in synchrony in the deterministic architecture. In addition, feedback fusion engine 936 can provide haptic robot feedback instructions to assist user movement so that activity requirements can be met.

Feedback fusion engine 936 can generate instructions for feedback devices 908 to feedback abstraction layer 938 which may have been compiled as part of the programs as described earlier herein such that specific feedback devices are synchronized. Further, it may act as an abstraction layer, or device driver, such that feedback instructions are translated to native instructions for the particular relevant hardware devices to realize the feedback. Feedback instructions preferrably also include parameters for the aspects of feedback. For example, for a haptic arm, instructions can include the type of movement based on physiological data extracted from the biosignal data. Feedback devices 908 can include various hardware actuators 940, visual feedback 942, other feedback devices as noted herein, and the like.

Fused data can be sent to a user state engine 934 where relevant user state indicators can be determined. For example, in a rehabilitation context, user motivation or engagement can be determined according to image data, eye tracking data, and other data that may indicate user engagement with a rehabilitation exercise. Eye tracking data that shows user eyes away from a display beyond a predetermined threshold, for example more than 2 seconds, can indicate that user engagement has fallen below an acceptable level. In that case, a user state indicator can be set to indicate as much and be returned to context extraction and processing engine 932 to adjust intent parameters or activity parameters (i.e., parameters that determine aspects of activities within the rehabilitation context such as activity type, difficulty, duration, and the like) in parameters 950. For example, eye tracking may indicate user engagement below an acceptable level (e.g., >2 seconds for 3 times during an activity) and, thus, an activity parameter can be set to set an activity difficulty level parameter to a higher difficulty and to set an intent parameter for creation of visual feedback in the form of display element when eye tracking sensor data indicates further disengagement. In that case, the activity difficulty rises according to the feedback loop and visual feedback is presented to maintain engagement. User states can include level of engagement (determined, for example, from sensor data showing reaction time, posture, eye tracking, and the like, using various sensors), emotional state (determined, for example, from sensor data showing eye tracking, galvanic skin response, reaction time, voice recognition, and the like, using various sensors), motivation (determined, for example, from activity history, reaction time, eye tracking, and the like, using various sensors) and so forth.

It should be understood that some or all of the system components illustrated in FIG. 9B can be further combined or separated, can be implemented as hardware, software, or a combination thereof, and can be implemented using the same or different hardware components.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A system for obtaining data about a subject, the system comprising a plurality of sensors for obtaining various types of data, a plurality of data consumers for consuming the data and a communication channel, wherein the sensors, data consumers and communication channel are arranged in a deterministic architecture.

2. The system of claim 1, wherein said communication channel supports a predetermined window function, in which different sensors transmit their data at a predefined window.

3. The system of claim 2, wherein said window is determined according to time, such that each sensor periodically has a set time period to transmit data.

4. The system of claim 3 wherein said window is determined according to a frequency division, such that each sensor transmits data only at a specific frequency.

5. The system of claim 1, wherein the sensors further comprise sensors for obtaining motion data in relation to the subject.

6. The system of claim 1, wherein each sensor is associated with a node, said node supporting data transmission from a particular sensor, said node comprising a processing unit for processing the data before transmission on the communication channel.

7. The system of claim 1, further comprising a data abstraction layer that abstracts the data before it is provided to the data consumers, to support communication between the sensors and the data consumers according to one or more predefined formats.

8. The system of claim 1, wherein said communication channel comprises a bus.

9. The system of claim 8 wherein the system is embodied as a chip.

10. The system of claim 1, implemented as a plurality of separate hardware components, wherein the communication channel operates across the plurality of separate hardware components.

11. The system of claim 10, wherein the communication channel operates across the separate hardware components according to a deterministic architecture, wherein communication between hardware components is determined according to a plurality of predetermined, deterministic functions.

12. The system of claim 11, wherein said deterministic functions include determining the window for communication, constraints on the components, requirements of the components, and programs which are to process and/or consume data.

13. A platform for controlling the process of obtaining data in real time from a plurality of sensors, said plurality of sensors including at least biosignal sensors, for consumption by one or more data consumers, the platform comprising a predetermined architecture and a plurality of predetermined functions for controlling said predetermined architecture.

14. The platform of claim 13, wherein said sensors communicate with the one or more data consumers through a predetermined communication channel, wherein the predetermined functions comprise functions for supporting communication through the communication channel.

15. The platform of claim 14, wherein the predetermined architecture determines the interactions of the sensors, the one or more data consumers and the communication channel, wherein the operating system defines the predetermined architecture.

16. The platform of claim 15, further comprising at the time of initiation of activity, determining a boot process, through which necessary programs are loaded, the communication channel is activated, and the sensors and data consumer(s) are able to communicate through the communication channel.

17. The platform of claim 16, wherein the communication channel supports a predetermined delay to support communication according to a predetermined timing.

18. The platform of claim 17, wherein the communication channel supports real time communication.

19. The platform of claim 18, wherein the communication channel supports an interrupt.

20. The platform of claim 13, wherein the sensors are selected from the group consisting of biosignal sensors, motion tracking sensors and additional subject related sensors.

21. The platform of claim 20, wherein said biosignal sensors comprise one or more of EEG, EKG, GVR (galvanic skin response), EMG, or audio sensors.

22. The platform of claim 21, wherein said motion tracking sensors include one or more of inertial sensors, IMU, optical sensors, one or more gyroscopes, one or more accelerometers, one or more magnetometers, or depth sensors.

23. The platform of claim 22, wherein said subject related sensors comprise optical sensors and/or complete sensor systems.

24. The platform of claim 23, wherein said complete sensor system comprises MindMotion Pro device of MindMaze SA (Lausanne, Switzerland).
